(19) United States
(12) Patent Application Publication
(10) Pub. No.: US 2018/0055490 A1
(43) Pub. Date: Mar. 1, 2018

(54) DEVICE AND METHOD FOR MANAGING PREGNANCY PLAN

(71) Applicant: BABYPLUS, Daejeon (KR)

(72) Inventors: Dong Hyoung LEE, Daejeon (KR); Moon Gyu KIM, Seongnam-si (KR); Ji Won SEO, Daejeon (KR); Gyu Ri MUN, Daejeon (KR); Shin Jee PYO, Daejeon (KR); Jun Hew CHO, Daejeon (KR)

(21) Appl. No.: 15/333,373

(22) Filed: Oct. 25, 2016

(30) Foreign Application Priority Data
Sep. 1, 2016 (KR) 10-2016-0112763

(51) Int. Cl. A61B 10/00; A61B 5/145; A61B 5/00

(52) U.S. Cl. CPC A61B 10/0012; A61B 5/14546; A61B 5/7455; A61B 5/7275; A61B 5/742; A61B 5/4306

(57) ABSTRACT

A device for managing pregnancy plan includes an ovulation tester configured to test whether an ovulation has taken place and provide a result of a ovulation test, and a mobile terminal configured to provide information related to ovulation to a user, wherein the mobile terminal includes a display, a memory configured to store basic information related to ovulation including at least recent start date of menstruation, and a processor configured to receive the result from the ovulation tester, to determine at least one of a predicted ovulation date and a ovulation date based on at least one of the basic information and the result, to determine a predicted fertility period therefrom, and to display the predicted fertility period on the display.

### CROSS-REFERENCE TO RELATED APPLICATIONS

Pursuant to 35 U.S.C. §119 (a), this application claims the benefit of earlier filing dates and rights of priority to Korean Patent Applications No. 10-2016-0112763, filed on Sep. 1, 2016, the contents of which are hereby incorporated by reference in their entirety.

### BACKGROUND OF THE INVENTION

### 1. Field of the Invention

The teachings in accordance with the exemplary embodiments of this present disclosure generally relate to a device and method for managing pregnancy plan.

### 2. Description of Related Art

The pregnancy plan means a variety of activities for intentionally inducing or avoiding pregnancy. The pregnancy may be scheduled by prediction of ovulation date by subtracting about 14 days from next start date of menstrual period or by using an ovulation tester.

The ovulation tester refers to a device configured to test whether ovulation has taken place by detecting bodily information such as hormone and body temperature, and by analyzing the detected information.

A method most widely used by the ovulation tester is to measure concentration of LH (Luteinizing Hormone). The LH shows a sudden change prior to or after the ovulation, whereby whether the ovulation has taken place can be ascertained by checking the concentration of the hormone.

Meantime, there are circumstances where an individual may have an insufficient knowledge on ovulation, and even if an individual has knowledge on the ovulation, it may be difficult to perform a systematic management on pregnancy plan because an ovulation period is different for each individual.

Furthermore, it is difficult to predict an ovulation date, because an ovulation tester provides only information of a particular date on whether ovulation has taken place, even if an individual uses the ovulation tester. On top of that, the ovulation tester suffers another drawback of increasing a burden of testing expenses if an accurate ovulation date cannot be predicted, because a test strip used for an ovulation tester is disposable or useable only once.

### SUMMARY OF THE INVENTION

The present disclosure is provided to solve the aforementioned disadvantage(s) and therefore, an exemplary embodiment of the present disclosure is to provide a device configured to perform a systematic management of pregnancy plan, and a method for managing the pregnancy plan.

Furthermore, an exemplary embodiment of the present disclosure is to provide a device for managing a pregnancy plan configured to enhance convenience and predictability by determining a predicted (estimated, expected) ovulation date, an ovulation date, an ovulation test date and fertility period, and by notifying the same to a user through a mobile terminal or an app of a smart phone, and a method for managing the pregnancy plan.

In order to achieve at least the above objects, in whole or in part, and in accordance with the purposes of the present disclosure, as embodied and broadly described, and in one general aspect, there may be provided a device for managing a pregnancy plan, the device comprising: an ovulation tester configured to test whether an ovulation has taken place and provide a result of a ovulation test; and a mobile terminal configured to provide information related to ovulation to a user; wherein the mobile terminal comprises: a display, a memory configured to store basic information related to ovulation including at least recent start date of menstruation, and a processor configured to receive the result from the ovulation tester, to determine at least one of a predicted ovulation date and a ovulation date based on at least one of the basic information and the result, to determine a predicted fertility period therefrom, and to display the predicted fertility period on the display.

In some exemplary embodiment of the present invention, the processor may further determine an ovulation test date within a preset date scope about the predicted ovulation date, and further displays the ovulation test date on the display when reaching the ovulation date.

In some exemplary embodiment of the present invention, the memory may further store a preset time, and the processor may display the ovulation test date at the preset time on the display when reaching the ovulation date.

In some exemplary embodiment of the present invention, the mobile terminal may further comprise a vibration unit, and the processor may operate the vibration unit when reaching the ovulation date.

In some exemplary embodiment of the present invention, the memory may further store a preset value an LH concentration, and the result comprises the LH concentration, and wherein the processor may adjust the predicted ovulation date when the LH concentration received on the very date is less than the preset value.

In some exemplary embodiment of the present invention, the memory may further store a preset value of an LH concentration, and the result may comprise the LH concentration, and wherein the processor may receive the result at least twice, and may determine the ovulation date through a comparison of the LH concentration of the previous day when the LH concentration received on the very date is more than the preset value.

In some exemplary embodiment of the present invention, the processor may calculate a predicted date error, which is a difference between the predicted ovulation date and the ovulation date, and determine a predicted ovulation date of next period by reflecting the predicted date error.

In some exemplary embodiment of the present invention, the processor may determine a start time of the predicted fertility period based on the predicted ovulation date prior to determination of the ovulation date, and determine an end time of the predicted fertility period based on the ovulation date after the determination of the ovulation date.

In another general aspect of the present invention, there may be provided a method for controlling a mobile device, the method comprising: receiving a result of an ovulation test from an ovulation tester; storing basic information related to ovulation including at least recent start date of menstruation in a memory of the mobile terminal; determining at least one of a predicted ovulation date and an ovulation date based on at least one of the basic information and the result; determining a predicted fertility period based on at least one of the predicted ovulation date and the ovulation date; and displaying the predicted fertility period on a display of the mobile terminal.

In some exemplary embodiment of the present invention, the method may further comprise: determining an ovulation test date within a preset date scope about the predicted ovulation date; and displaying the ovulation test date on the display when reaching the ovulation date.

In some exemplary embodiment of the present invention, the method may further comprise: storing a preset time; and displaying the ovulation test date on the display at the preset time when reaching the ovulation date.

In some exemplary embodiment of the present invention, the result may comprise an LH concentration, and the method may further comprise: adjusting the predicted ovulation date when an LH concentration received on the very date is less than the preset value.

In some exemplary embodiment of the present invention, the result may comprise an LH concentration, and the method may further comprise: storing a preset value of the LH concentration; receiving the result at least twice; and determining the ovulation date through a comparison of the LH concentration of the previous day when the LH concentration received on the very date is more than the preset value.

In some exemplary embodiment of the present invention, the method may further comprise: calculating a predicted date error, which is a difference between the predicted ovulation date and the ovulation date; and determining a predicted ovulation date of next period by reflecting the predicted date error.

In some exemplary embodiment of the present invention, a start time of the predicted fertility period may be determined based on the predicted ovulation date prior to determination of the ovulation date, and an end time of the predicted fertility period may be determined based on the ovulation date after the determination of the ovulation date.

### Advantageous Effects of the Disclosure

The present disclosure has an advantageous effect in that a systematic management of pregnancy plan can be performed.

Another advantageous effect of the present disclosure is that costs required for ovulation test can be reduced through adjustment of predicted ovulation date.

Still another advantageous effect of the present disclosure is that the ovulation date is shared to allow ovulation-related information to be shared along with a spouse.

### BRIEF DESCRIPTION OF THE DRAWINGS

FIG. 1 is a schematic diagram illustrating a device for managing pregnancy plan according to an exemplary embodiment of the present disclosure.

FIG. 2 is a flowchart illustrating a method for managing pregnancy plan according to an exemplary embodiment of the present disclosure.

FIG. 3 is a flowchart illustrating a method for adjusting a predicted ovulation date according to an exemplary embodiment of the present disclosure.

FIG. 4 is a flowchart illustrating a method for determining an ovulation date according to an exemplary embodiment of the present disclosure.

### DETAILED DESCRIPTION

The present disclosure may be applied with various changes, and may be included with various exemplary embodiments, and particular exemplary embodiments will be exemplified by drawings and explained in the Detailed Description. However, the present disclosure will not be limited to the particular exemplary embodiments, and the described aspect is intended to embrace all such alterations, modifications, and variations that fall within the scope and novel idea of the present invention.

Accordingly, in some embodiments, well-known processes, well-known device structures and well-known techniques are not illustrated in detail to avoid unclear interpretation of the present disclosure.

Hereinafter, exemplary embodiments of the present disclosure will be described in detail with reference to the accompanying drawings.

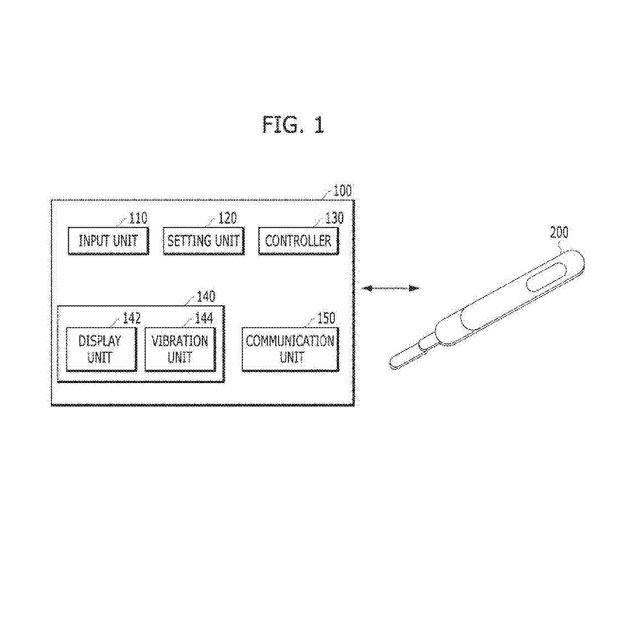

FIG. 1 is a schematic diagram illustrating a device (100) for managing pregnancy plan according to an exemplary embodiment of the present disclosure.

Referring to FIG. 1, the device (100) for managing pregnancy plan according to an exemplary embodiment of the present disclosure may include an input unit (110), a setting unit (120), a controller (130), a notifying unit (140), a communication unit (150) and storage (160).

The device (100) for managing pregnancy plan (hereinafter simply referred to as device 100) according to an exemplary embodiment of the present disclosure may be a smart phone installed with an application recorded with a program of a method for managing pregnancy plan according to an exemplary embodiment of the present disclosure. However, the present disclosure is not limited thereto. For example, the device (100) for managing pregnancy plan may be a device manufactured solely for management of pregnancy plan recorded and embedded with a program of a method for managing pregnancy plan according to an exemplary embodiment of the present disclosure.

The input unit (110) may receive basic information related to ovulation. The basic information related ovulation may mean information for determining a predicted ovulation date and may include a recent menstrual period and menstrual cycle. The input unit (110) may be configured with an interface such as a keypad or a touch screen for receiving the said information from a user.

The setting unit (120) may be configured to set various operation conditions or methods for the device (100) for managing pregnancy plan. The setting of the setting unit (120) may be directly performed by the controller (130), or may be also directly set by a user through a UI (User Interface).

The setting unit (120) may set an ovulation date, a notifying method of a predicted ovulation period, a notifying time, or information of whether to share with a spouse's terminal. However, when these items are not set up, the controller (130) may be so programmed as to automatically set up these items.

Here, the spouse's terminal may mean a terminal such as a smart phone used by a person who desires to share the information. The terminal of a person who desires to share the information with regard to pregnancy plan may be generally a terminal used by a spouse (husband).

The controller (130) may function to determine an ovulation date and a predicted ovulation date, and to calculate an error of predicted ovulation date. The controller (130) may calculate a predicted fertility period using the ovulation date and a predicted ovulation date.

The predicted fertility period may be calculated as a period from 5 days before a determined ovulation date to 2 days after the determined ovulation date. However, the exemplary embodiments of the present disclosure are not limited thereto. For example, before determination of ovulation date, a predicted fertility period may be calculated as a period from 5 days before a determined ovulation predicted date to 2 days after the determined ovulation predicted date, and after determination of ovulation date, a predicted fertility period may be also calculated as a period from 5 days before a determined ovulation date to 2 days after the determined ovulation date. In this case, the predicted ovulation date of ovulation date may be changed depending on period, and as a result, the predicted fertility period may be also changed.

A fertility period is a period when pregnancy possibility is high, and is generally considered 5 days before the ovulation, and 2 days after ovulation.

The controller (130) may control operations of the setting unit (120), the notifying unit (140) and the communication unit (150).

The notifying unit (140) functions to notify to a user various pieces of information determined or calculated by the device (100) by way of visual, auditory and/or tactile methods. The notifying unit (140) may include a display (142) and a vibration unit (144). The display (142) may be a screen of a smart phone. The display (142) may visually display information related to pregnancy plan to allow a user to recognize the information.

The vibration unit (144) may tactually notify the relevant information to a user. The vibration unit (144) may be effective in notifying that a particular data or a particular time has arrived. For example, a pushing and notifying method is widely used in case of smart phone, and a notifying method through vibration is largely used among other methods. That is, when a smart phone is used as or for the device (100), the device (100) may be formed by a vibration unit of a smart phone.

The communication unit (150) may exchange information through communication with the ovulation tester (200) and a terminal of a spouse. Although the communication unit (150) may also directly communicate with the terminal of a spouse, communication may be performed by an indirect method in which information is transmitted to a server, and the server in turn transmits the information to a terminal of a spouse.

The storage (160) may store basic information and set contents of the setting unit (120). That is, the storage (160) may store a last date of menstrual period, menstrual cycle, a notifying method of ovulation date and predicted ovulation date and a notifying time thereof.

The ovulation tester (200) may be a device that measures concentration of user's LH, and transmits information including the LH concentration to the device (100). Although the ovulation tester (200) can transmit information through LAN (Local Area Network) such as Bluetooth, the exemplary embodiments of the present disclosure are not always limited thereto. For example, the device (100) and the ovulation tester (200) may also exchange information through WI-Fi connection.

The information measured and obtained by the ovulation tester (200) in the present invention may not be always limited to the LH concentration as in the exemplary embodiments of the present disclosure. For example, the ovulation tester (200) may measure any one of HCG (Human Chorionic Gonadotropin), FSH (Follicle-Stimulating Hormone), estradiol and progesterone, and may transmit information corresponding thereto to the device (100).

The communication unit (150) may communicate with a server providing applications recorded in a program by the method for managing pregnancy plan. In this case, the device (100) may download the application through the communication unit (150).

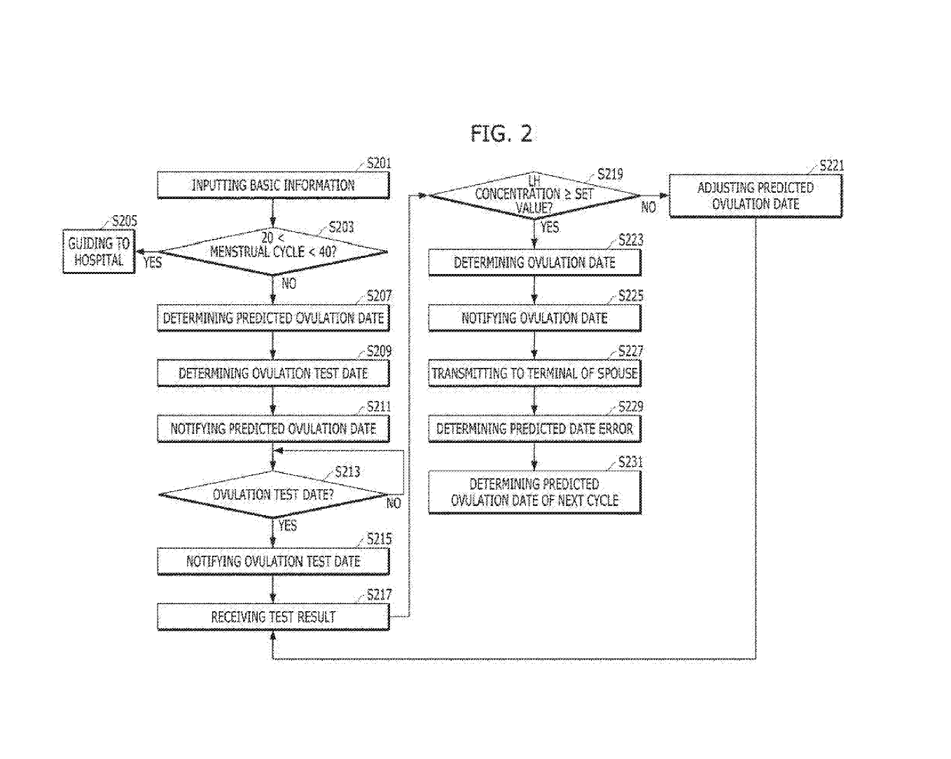

FIG. 2 is a flowchart illustrating a method for managing pregnancy plan according to an exemplary embodiment of the present disclosure.

The method for managing pregnancy plan according to an exemplary embodiment of the present disclosure may be performed by the device (100) thus explained. Now, the method for managing pregnancy plan according to an exemplary embodiment of the present disclosure through operations of the device (100) will be described in detail.

Referring to FIG. 2, the input unit (110) may receive basic information at step S201.

The controller (130) at step S203 may determine whether the menstrual cycle exceeds 20 days, and the menstrual cycle is less than 40 days. When the menstrual cycle is not within the above scope, the process advances to step S205, and when the menstrual cycle is within the scope, the process advances to step S207. This type of operation by the controller (130) is to notify a relevant measure when the menstrual cycle is irregular by determining whether the menstrual cycle is regular or irregular. Thus, there is no need that the scope be always limited to 20 days and 40 days.

The notifying unit (140) at step S205 may notify to the user information about a hospital. To be more specific, the notifying unit (140) may notify to the user information about a nearest hospital. However, the exemplary embodiment of the present disclosure is not limited thereto. For example, instead of information on hospitals, the display (142) may be displayed with information including a piece of advice when the menstrual cycle is irregular.

The controller (130) at step S207 may determine the predicted ovulation date using the basic information. The controller (130) may determine a next predicted menstrual date by adding a recent menstrual date with the menstrual cycle, and determine the predicted ovulation date by subtracting 14 days from the predicted menstrual date. The determination of predicted ovulation date may be expressed by the following Equation 1:

Predicted ovulation date=recent menstrual date+menstrual cycle−14 [Equation 1]

The controller (130) at step S209 may determine an ovulation test date. The ovulation test date may be determined within a preset date scope. Although the date scope may be set by a user through the setting unit (120), 3 days before and after the predicted ovulation date are determined as the ovulation test date according to the exemplary embodiment of the present invention. That is, the ovulation test date may be a period of a total 7 days including the predicted ovulation date. However, the exemplary embodiment of the present disclosure is not limited thereto. When the prediction (estimation) of predicted ovulation dale is more accurate than the prediction, the controller (130) may also determine 2 days before and alter the predicted ovulation dale, or one day before and after the predicted ovulation date as the ovulation test date.

Of course, if it is determined by a user that there is no further obtainable information after obtaining a desired piece of information, test may be performed only on some dates among the ovulation test date.

Furthermore, the controller (130) may determine a start time of predicted fertility period using the predicted ovulation date. The start time of predicted fertility period may be expressed by the following Equation 2:

Start time of predicted fertility period=predicted ovulation date−5 days [Equation 2]

The notifying unit (140) at step S211 may notify the predicted ovulation date to a user. The predicted ovulation date may be preferable to be visually displayed through the display (142).

The controller (130) at step S213 may determine whether the very day (today) is the ovulation test date. When it is determined that today is the ovulation test date, the process may advance to step S215, and repeat the step S213 when it is not determined that today is the ovulation test date.

The notifying unit (140) at step S215 may notify to the user that today is the ovulation test date. When the device (100) uses a smart phone and application thereof, a method of notifying the ovulation test date may include using letters, push notification and/or vibration notification. An operation of notifying the ovulation test date may be performed only on a first ovulation test date, and may be also performed every day during the ovulation test date (7 days in the exemplary embodiment of the present disclosure).

The notification of ovulation test date may be preferable by matching to the ovulation test period. For example, when it is preferable to perform the ovulation test during 10:00 hours to 20.00 hours, the notifying unit (140) may notify to a user that today (the very day) is the ovulation test date, and that it is preferable to perform the test during 10:00 hours to 20.00 hours.

The communication unit at step S217 may receive a test result from the ovulation tester (200). The test result may include an LH concentration, test date and test time.

The controller at step S219 may determine whether the LH concentration is greater than a preset value. Although the preset value is set at 25 miu according to the exemplary embodiment of the present disclosure, the preset value is not limited thereto. For example, the present value may be also set at 20 miu and 30 miu. The numerical value change of hormone may be variable according to each person, and numerical value change of hormone may be also different depending on ages, such that a set value may be differently established depending on circumstances.

The set value may be also calculated by a preferable value using a user's age, menstrual period, existing test result information, and a user may directly determine the set value through the setting unit (120).

When the LH concentration is above a preset value at step S219, the process may advance to step S223, and when the LH concentration is less than a preset value at step S219, the process may advance to step S221.

The controller (130) may adjust a predicted ovulation date at step S221. The reason of adjusting the predicted ovulation date is to change the predicted ovulation date to a date later than an initially determined predicted ovulation date, because the LH concentration is not measured at a value higher than a set value even on the ovulation test date.

The controller (130) may determine an ovulation date at step S223. When the LH concentration is measured at a value higher than the set value, the controller (130) may come to have a condition capable of determining the ovulation date because candidates of ovulation dates are very much narrowed.

The notifying unit (140) may notify user the determined ovulation date to a user at step S225. The controller (130) may determine an end point of predicted fertility period using the ovulation date. The end time of the predicted fertility period may be determined by the following Equation 3:

End time of predicted fertility period=ovulation date+2 days [Equation 3]

That is, a start time of predicted fertility period according to an exemplary embodiment of the present disclosure may be determined based on the predicted ovulation date, and the end time of the predicted fertility period may be determined based on an ovulation date determined as a result of tests tested by the ovulation tester. As discussed above, there is a possibility of the ovulation date being changed in response to repeated tests, and as a measure thereto, the end time of the predicted fertility period may be determined by being changed.

The notifying unit (140) may notify the predicted fertility date to the user.

The characteristic aspect of the present disclosure is that prediction (estimation) of predicted ovulation date and actual ovulation test have been combined. That is, although only the prediction of predicted ovulation date may learn a time (timing) of menstrual cycle, there is no way of knowing an actual state of a user herself, and although only the ovulation test may learn an actual state of a user herself, there is no way of knowing which time of state the actual state is on the menstrual cycle. For example, the result of ovulation test alone cannot learn whether the state is within the fertility period, the state is before the ovulation or the state is after the ovulation, even if a result of no ovulation is obtained.

The present disclosure can notify information on the fertility period, and when a determined time arrives, the present disclosure can notify behaviors such as ovulation test to be performed by a user, and can notify by flexibly changing the predicted fertility period in response to the test result, through which a user can be accomplished with an effect of simultaneously grasping her (principal's) state and at which position the her own (principal's) state is situated in the menstrual period.

Furthermore, the effect is that the present invention may be realized by a mobile terminal or an application of a smart phone, and her own (principal's) state can be monitored at all times.

The communication unit (150) at step S227 may transmit the determined ovulation date to a terminal of a spouse, which is to assist in performance of pregnancy plan with a spouse by sharing information related to the ovulation date.

The controller (130) at step S229 may determine a predicted date error. The predicted date error is a difference between an initially determined predicted ovulation date and a determined ovulation date. However, the present disclosure is not limited thereto. For example, the predicted date error may be a difference between initial predicted ovulation date and lastly adjusted predicted ovulation date, and may be also an average value of accumulated differences between ovulation dates and predicted ovulation date.

The controller (130) at step S231 may determine a next predicted ovulation date.

The controller (130) in the exemplary embodiment of the present disclosure may determine the next cycle of predicted ovulation date by adding up a user's ovulation cycle from next menstrual start day, subtracting therefrom by 14 days, and by adding the predicted date error.

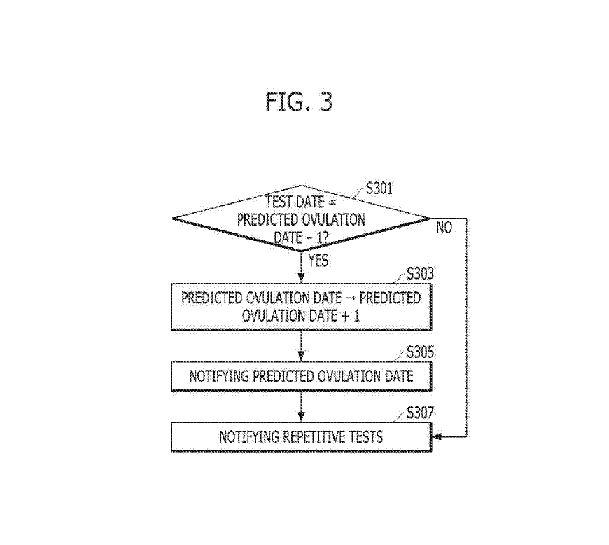

FIG. 3 is a flowchart illustrating a method for adjusting a predicted ovulation date according to an exemplary embodiment of the present disclosure. That is, FIG. 3 is a flowchart for explaining the step S221 in more detail.

Referring to FIG. 3, the controller (130) at step S301 may check whether the very date of test is one day before the predicted ovulation date. If the very date of test is one day before the predicted ovulation date, the process advances to S303, and if the very date of test is not one day before the predicted ovulation date, the process advances to S307.

The controller (130) at step S303 may change the predicted ovulation date to a date in which one day is added to the initially determined predicted ovulation date, which is due to the fact that ovulation may be later (or delayed) than the predicted ovulation date when the LH concentration shows no increase in numerical value even though the date is a very day prior to the predicted ovulation date.

The notifying unit (140) at step S305 may notify the adjusted predicted ovulation date to a user. The controller (130) may transmit the adjusted predicted ovulation date to a terminal of a spouse by controlling the communication unit (150).

The notifying unit (140) at step S307 may notify that the test must be repetitively performed even on the next day. The notifying unit (140) may notify that right after the test, or may notify at a preferable test time of next day.

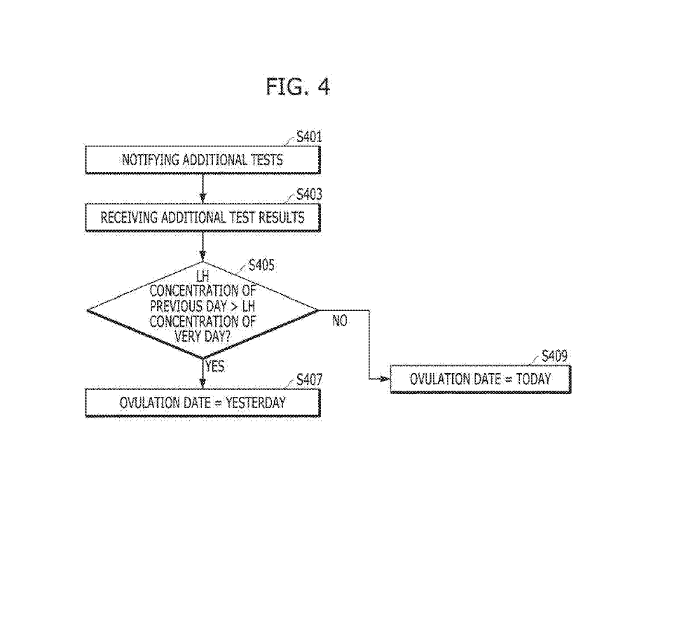

FIG. 4 is a flowchart illustrating a method for determining an ovulation date according to an exemplary embodiment of the present disclosure. That is, FIG. 4 is a flowchart for explaining the step S223 in more detail.

Referring to FIG. 4, the notifying unit (140) at step S401 may notify to a user that the ovulation test must be performed additionally once more. When the LH concentration exceeds 25 miu, there is a high probability that the ovulation may take place next day. Thus, performance of one more test may learn the ovulation date more accurately.

The communication unit (150) at step S403 may receive the ovulation test result additionally.

The controller (130) at step S405 may check whether the LH concentration received a day before is greater than the LH concentration on the very day. If the LH concentration received a day before is greater than the LH concentration on the very day, the process advances to S407, and if the LH concentration received a day before is not greater than the LH concentration on the very day, the process advances to S409.

The controller (130) at step S407 may determine a day before the very day as the ovulation date. This is because the LH concentration shows a highest numerical value on the ovulation date, and drops abruptly thereafter.

The controller (130) at step S409 may determine the very day as the ovulation date. This is because the day has a high possibility, in most cases, of the ovulation having taken place, when the LH concentration on a next day, where the LH concentration was measured at more than 25, becomes greater than 25.

However, the process as in FIG. 4 is not required at all times. When the LH concentration is measured over 25 miu, it may be determined that the ovulation has taken place right way, such that the day may be determined as an ovulation date, and the ovulation date may be determined by additionally receiving test results of more than two days. The numerical value of LH concentration based on ovulation may also differ depending on ages, such that the level of LH concentration determining the ovulation date may be also set up.

As discussed in the foregoing, although all the elements forming the exemplary embodiments of the present disclosure are combined into one or operated as one element, the present disclosure is not limited thereto. That is, all the elements may be selectively combined or operated if within an object scope of the present disclosure. Furthermore, it will be understood that the terms “includes” and/or “including”. “forming” and/or “formed” when used in this specification, specify the presence of stated features, regions, integers, steps, operations, elements, and/or components, but do not preclude the presence or addition of one or more other features, regions, integers, steps, operations, elements, components, and/or groups thereof.

What is claimed is:

1. A device for managing a pregnancy plan, the device comprising:
an ovulation tester configured to test whether an ovulation has taken place and provide a result of a ovulation test; and
a mobile terminal configured to provide information related to ovulation to a user; wherein the mobile terminal comprises:
a display,
a memory configured to store basic information related to ovulation including at least recent start date of menstruation, and
a processor configured to receive the result from the ovulation tester, to determine at least one of a predicted ovulation date and a ovulation date based on at least one of the basic information and the result, to determine a predicted fertility period therefrom, and to display the predicted fertility period on the display.

2. The device of claim 1, wherein the processor further determines an ovulation test date within a preset date scope about the predicted ovulation date, and further displays the ovulation test date on the display when reaching the ovulation date.

3. The device of claim 2, wherein the memory further stores a preset time, and the processor displays the ovulation test date at the preset time on the display when reaching the ovulation date.

4. The device of claim 2, wherein the mobile terminal further comprises a vibration unit, and the processor operates the vibration unit when reaching the ovulation date.

5. The device of claim 1, wherein the memory further stores a preset value an LH concentration, and the result comprises the LH concentration,
wherein the processor adjusts the predicted ovulation date when the LH concentration received on the very date is less than the preset value.

6. The device of claim 1, wherein the memory further stores a preset value of an LH concentration, and the result comprises the LH concentration,
wherein the processor receives the result at least twice, and determines the ovulation date through a comparison of the LH concentration of the previous day when the LH concentration received on the very date is more than the preset value.

7. The device of claim 6, wherein the processor calculates a predicted date error, which is a difference between the predicted ovulation date and the ovulation date, and determines a predicted ovulation date of next period by reflecting the predicted date error.

8. The device of claim 6, wherein the processor determines a start time of the predicted fertility period based on the predicted ovulation date prior to determination of the ovulation date, and determines an end time of the predicted fertility period based on the ovulation date after the determination of the ovulation date.

9. A method for controlling a mobile device, the method comprising:
receiving a result of an ovulation test from an ovulation tester;
storing basic information related to ovulation including at least recent start date of menstruation in a memory of the mobile terminal;
determining at least one of a predicted ovulation date and an ovulation date based on at least one of the basic information and the result;
determining a predicted fertility period based on at least one of the predicted ovulation date and the ovulation date; and
displaying the predicted fertility period on a display of the mobile terminal.

10. The method of claim 9, further comprising:
determining an ovulation test date within a preset date scope about the predicted ovulation date; and
displaying the ovulation test date on the display when reaching the ovulation date.

11. The method of claim 10, further comprising:
storing a preset time; and
displaying the ovulation test date on the display at the preset time when reaching the ovulation date.

12. The method of claim 9, wherein the result comprises an LH concentration, and the method further comprises:
adjusting the predicted ovulation date when an LH concentration received on the very date is less than the preset value.

13. The method of claim 9, wherein the result comprises an LH concentration, and the method further comprises:
storing a preset value of the LH concentration;
receiving the result at least twice; and
determining the ovulation date through a comparison of the LH concentration of the previous day when the LH concentration received on the very date is more than the preset value.

14. The method of claim 13, further comprising:
calculating a predicted date error, which is a difference between the predicted ovulation date and the ovulation date; and
determining a predicted ovulation date of next period by reflecting the predicted date error.

15. The method of claim 13, wherein a start time of the predicted fertility period is determined based on the predicted ovulation date prior to determination of the ovulation date, and an end time of the predicted fertility period is determined based on the ovulation date after the determination of the ovulation date.
